(19) United States
(12) Patent Application Publication
(10) Pub. No.: US 2010/0222807 A1
(43) Pub. Date: Sep. 2, 2010

(54) Method of stabilizing an arthritic carpometacarpal joint of the thumb

(76) Inventor: Sue Ellen Martin, South Pasadena, CA (US)

Correspondence Address: Sue Ellen Martin, 1634 Spruce Street, South Pasadena, CA, 91030, US

(21) Appl. No.: 12/380,454

(22) Filed: Feb. 27, 2009

(51) Int. Cl. A61B 17/00

(52) U.S. Cl. 606/201; 128/898

(57) ABSTRACT

The use of an elastic band across the hand that selectively prevents any outward displacement (subluxation) of the metacarpalcarpal (Trapeziometacarpal) joint of the thumb is described as a method of reducing the symptoms of arthritis of this joint and its attendant atrophy. This method has many advantages over the existing larger hand splints commonly prescribed for thumb arthritis. Specifically, it allows for a greater range of motion of the hand, including the thumb than do the presently commercially available hand splints. It is also ascetically preferable and more hygienic than these alternative splints.

### CROSS REFERENCE TO RELATED AWARDED AND TO PENDING PATENS

Smith S A. One piece thumb support and protector. U.S. Pat. No. 4,658,441

Theisler C W. Adjustable thumb brace. U.S. Pat. No. 4,953,568

Colditz J C. Thumb CMC restriction splint. U.S. Pat. No. 6,702,772

Fisher H. Thumb splint. U.S. Pat. No. 6,783,507

Fisher H. Bandage for the prevention of radial luxation of the thumb Application 200402101

### STATEMENT REGARDING FEDERALLY SPONSORED RESEARCH OR DEVELOPMENT

No Federal funding was received in support of this patent application.

### REFERENCE TO SEQUENCE LISTING, A TABLE, OR A COMPUTER PROGRAM LISTING COMPACT DISK APPENDIX

None provided.

### BACKGROUND OF THE INVENTION

The ability to oppose the thumb to other fingers of the hand involves greater mobility of the carpometacarpal joint of the thumb compared with that of the other fingers. (This joint is also referred to as the trapeziometacarpal joint and will be hereinafter abbreviated as TMJ.) It is the rotational as well as the back and forth movements of the thumb that results in cartilage damage to the TMJ that exceeds that occurring in the carpometacarpal joints of the other fingers. Moreover, the capsule with its five thickened bands (ligaments) that stabilizes the TMJ is relatively weak and, in woman, can become even weaker as estrogen levels fall. Consequently, symptomatic degeneration (arthritis) of the TMJ is particularly frequent in post menopausal women with an incidence variously estimated at approximately 25%. Various sporting and occupational activities that involve excessive stress on the thumb, such as basketball playing and cutting hair, can also promote the development of thumb arthritis, as can rheumatoid changes and other connective tissue disorders including lupus and Elhers Danlos syndrome. The most prominent symptoms of TMJ arthritis are pain; swelling from inflammation and exophyte (bony outgrowth) formation; weakness in hand grasping of objects with muscle atrophy leading to further restriction of mobility and dexterity.

Complex hand splints are commonly prescribed to alleviate the pain and other symptoms of TMJ arthritis. These splints are primarily aimed at an overall restriction of the movements of the thumb, primarily by bracing the thumb to the index finger with a portion of the splint passing between the thumb and index finger. Most of the currently marketed splints extend across the whole hand and typically also embrace the wrist. Such splints are not particularly effective or convenient. Thus many patients remain on pain medications and actual hand surgery on the affected joint is not uncommonly required to achieve tolerable relief from the increasingly worsening symptoms. Much of the progressive deterioration that typically occurs in patients wearing conventional hand splints is caused by the restricted movements imposed by the splints, which cause additional muscle atrophy. Furthermore, the hand splints are unsightly and embarrassing to many of their users and these factors tend to reduce compliance. There is, therefore, a need for an improved method to alleviate symptoms and help prevent further progression of TMJ arthritis. This patent application describes an extremely simple, ascetically acceptable, device to achieve these goals.

### BRIEF SUMMARY OF THE INVENTION

The invention is based on the inventor's understanding that the major functional deficit caused by TMJ arthritis is the inability of the joint's ligaments to prevent an outward displacement (subluxation) of the proximal head of the metacarpal bone The subluxation occurs in relationship to the underlying trapezium carpal bone, especially when the thumb is being rotated. This subluxation leads to symptoms such as pain and impaired functioning of the thumb and contributes to further aggravation of existing joint damage through induced inflammation and cartilage friction.

The invention is a remedy for arthritis of the thumb that comprises a relatively narrow elastic band. The band is designed to specifically restrict the outward displacement (subluxation) of the proximal head of the metacarpal bone in relationship to the underlying trapezium carpal bone, as the thumb is being rotated. The elastic band wraps around the hand such that it selectively and specifically crosses the thumb at the level of the proximal head of the thumb metacarpal bone. The elastic band extends around the palm and back of the hand crossing the lower metacarpals of the other four fingers The preferable degree of constraint provided by the elastic band is that which is sufficient and adequate to achieve symptomatic relief, while still allowing for full rotator and other movement of the thumb, as well as full hand movements. Thus, the only restriction on the thumb is the limited pressure exerted on the proximal head of the first metacarpal bone required to prevent any outward displacement as the thumb is rotated. The continued normal use of the thumb avoids further atrophy and, indeed, allows for muscle strengthening exercises. Full adduction of the thumb is possible since the elastic band does not intercede across the space between the thumb and index finger, The elastic band invention has many additional aesthetic and hygienic benefits over the currently commercialized much larger hand splints.

### BRIEF DESCRIPTION OF THE DRAWINGS

None included

### DETAILED DESCRIPTION OF THE INVENTION

The invention comprises an elastic band that is simply wide enough (approximately 1-2 cm) to cover the proximal head of the thumb's metacarpal bone and long enough to fit comfortably and securely horizontally around the hand. The degree of tension exerted by the band is sufficient to prevent any outward displacement (subluxation) of the TMJ upon rotation of the thumb onto the base of the small (fifth) finger of the affected hand. The lack of subluxation can be determined by palpation and also by the elimination of pain that would otherwise occur in effecting this movement in the absence of the elastic band. The effectiveness of the band can also be assessed by the pain-free strength that can be exerted by the rotated thumb when pressed against the small (fifth) finger. While a typical broad rubber band that is light brown in color can be used for the purpose of this invention, it is envisioned that bands will be made available in a wider variety of colors to better match all skin colors. An elastic band that is somewhat wider in the section that will cover the head of the first metacarpal, for example 2 cm with a narrower width of <1 cm in the remaining portion, is also envisioned. As a further alternative two bands may be used that are spread wider apart at the head of the metacarpal bone but overlay elsewhere on the hand. A variety of band sizes will be available to allow fittings that exert the minimal adequate force on the head of the first metacarpal bone for maximum functional improvement and symptom reduction and prophylactic arthritis prevention. The bands can be disposable, easily washable and waterproof. They can be made from rubber or other elastic materials.

The invention has been tried under various clinical conditions and compared with several different types of currently commercialized hand splints. The design has been compared and contrasted with those described in other patent applications for TMJ arthritis and found to provide the above stated advantages. The design is also different from rigid hand splints primarily designed to prevent traumatic dislocations and subluxations of the TMJ (re: pending patent application 20040210179.)

The regular use of an elastic band has indeed led to improved functioning of the thumb in the grasping of objects and in the reversal of atrophy of the thenar muscles in a typical patient. Indeed, the patient now finds herself frequently exercising her thenar muscles without any attendant pain. The prospect of ever requiring surgery for progression of her thumb arthritis has clearly been reduced.

The principles, preferred embodiments and modes of operation of the present invention have been described in the foregoing specification. The invention which is intended to be protected herein, however, is not to be construed as limited to the particular form disclosed, since this is to be regarded as illustrative rather than restrictive. Additional advantages and modifications will readily occur to those skilled in the art.

What I claim as my invention is:

1. A method for alleviating the symptoms of arthritis of the carpometacarpal joint of the thumb comprising an elastic band that is placed around the hand in a manner to apply sufficient localized pressure to the proximal head of the metacarpal bone of the thumb to selectively restrict its outward displacement (subluxation) during hand movements involving the thumb.

2. The method of claim 1 in which the symptoms comprise one or more of the following: pain at rest and/or on moving the thumb, restricted ability and/or strength in opposing the thumb against other fingers of the hand, weakness of the hand in grasping objects and ability of using the thumb to perform fine movements.

3. A method of avoiding the likelihood of surgery being performed in the symptomatic relief of the symptoms of arthritis of the carpometacarpal joint of the thumb comprising an elastic band that is placed around the hand in a manner to apply sufficient localized pressure to the proximal head of the metacarpal bone of the thumb to restrict its outward displacement (subluxation) during hand movements involving the thumb.

4. A method of stabilizing the carpometacarpal joint of the thumb comprising an elastic band that is placed around the hand in a manner to apply sufficient localized pressure to the proximal head of the metacarpal bone of the thumb to selectively restrict its outward displacement (subluxation) during hand movements involving the thumb, while having no or only very minimal restriction on the movements of other fingers or palm of the hand.
